(19) United States
(12) Patent Application Publication
(10) Pub. No.: US 2023/0346673 A1
(43) Pub. Date: Nov. 2, 2023

(54) PHOTOCHEMICAL HAIR STYLING METHODS AND RELATED COMPOSITIONS

(71) Applicant: Virtue Labs, LLC, Winston-Salem, NC (US)

(72) Inventors: Erin FALCO, Winston-Salem, NC (US); Tiffany HENDLEY, Greensboro, NC (US); William JACOBSEN, Clemmons, NC (US)

(21) Appl. No.: 18/140,665

(22) Filed: Apr. 28, 2023

Related U.S. Application Data
(60) Provisional application No. 63/335,792, filed on Apr. 28, 2022.

(51) Int. Cl. A61K 8/65; A61Q 5/06

(52) U.S. Cl. CPC A61K 8/65; A61Q 5/06; A61K 2800/81; A61K 2800/805; A61K 2800/95

(57) ABSTRACT

A hair styling kit is provided that includes a keratin protein composition and an activator composition. The keratin protein composition and activator composition may be applied to hair to increase manageability of hair, reduce frizz, and relax curl. Methods of treatment are also provided.

### CROSS-REFERENCE TO RELATED APPLICATIONS

The present application claims priority to U.S. Provisional Application No. 63/335,792 filed Apr. 28, 2022.

### BACKGROUND

Keratins are fundamental proteins of the skin, the hair, the eyelashes and the nails. These fibrous proteins contribute towards their form, elasticity and strength. Human hair is a unique source of human keratins because hair is one of the few human sources that are readily available and inexpensive.

Cosmetic treatments of hair substantially deteriorate and weaken the hair as well as destroy the internal and external structures of the hair. Specifically, current practices of changing the texture of the hair all use chemicals and high temperatures that are harsh to both hair and scalp. These chemicals, when applied and heated, pose a health risk to both the user and the stylist that may be applying the chemicals to the user. Such chemicals include various organic compounds such as formaldehydes or other irritant chemicals such as sodium hydroxide. In fact, in California, new legislation seeks to ban certain cosmetic chemicals such as mercury, phthalates, parabens, and fluoroalkyl compounds. These harsh cosmetic chemicals often result in high pH and high temperature that significantly damage hair in order to obtain the desired styling results. As a result, the hair may become frizzy, brittle with split ends or the hair may break. Further, hair constituent proteins may be removed by treatments with cosmetics such as shampoos, perm chemicals, or hair dyes. As a result, the likelihood of hair damage increases as hair is unable to restore itself to its original state without assistance. Thus, there remains a need for compositions and styling methods that address these issues.

### SUMMARY

According to one aspect, a hair styling kit is provided. The kit includes: a keratin protein composition that includes alpha keratin, gamma keratin, or a combination thereof; and an activator composition that includes a co-initiator compound and a crosslinker compound. Upon exposure to light, the photochemical composition is activated. Upon activation, the hair may attain the desired texture by adjusting one or more of: (i) light wavelength; (ii) light intensity; (iii) temperature; (iv) duration of light exposure; (v) amount and length of time the activator composition remains in the hair; (vi) weight percent of components included in the keratin protein composition; and (vii) weight percent of components included in the activator composition. According to one embodiment, the keratin protein composition further includes ethylenediaminetetraacetic acid (EDTA) present in an amount of from about 0.01% w/w to about 3% w/w based on the total weight of the protein composition. According to one embodiment, the co-initiator compound is sodium persulfate, L-arginine, or a combination thereof. According to one embodiment, the crosslinker compound is riboflavin-5-phosphate. According to one embodiment, the alpha keratin, gamma keratin, or combination thereof is present in the protein composition in an amount of about 0.01% w/w to about 10% w/w based on the total weight of the keratin protein composition. According to one embodiment, the crosslinker compound is present in the activator composition in an amount of from about 0.01% w/w to about 10% w/w based on the total weight of the activator composition. According to one embodiment, the co-initiator compound is present in the activator composition in an amount of from about 0.01% w/w to about 30% w/w based on the total weight of the activator composition. According to one embodiment, the protein composition includes alpha keratin and gamma keratin. According to one embodiment, the alpha keratin to gamma keratin ratio in the keratin protein composition is about 4:1.

According to one aspect, a method of photochemical hair treatment is provided. The method of photochemical hair treatment includes the steps of: administering a keratin protein composition including alpha keratin, gamma keratin, or a combination thereof, the administration resulting in keratin protein treated hair; administering to a surface of the keratin protein treated hair an activator composition including a co-initiator compound and a crosslinker compound to produce activator composition treated hair; and exposing the activator composition treated hair to a light source. According to one embodiment, the method further includes the step of: adjusting one or more of: (i) light wavelength; (ii) light intensity; (iii) temperature; (iv) duration of light exposure; (v) amount and length of time the activator composition remains in the hair; (vi) weight percent of components included in the keratin protein composition; and (vii) weight percent of components included in the activator composition. According to one embodiment, the hair is exposed to the light source for at least one minute. According to one embodiment, the light source emits a light having a wavelength of from about 450 nm to about 550 nm. According to one embodiment, the light source exhibits an intensity of at least about 25,000 lux. According to one embodiment, the activator composition treated hair is exposed to a temperature of from about 25° C. to about 95° C.

According to one aspect, a method of relaxing hair or reducing curl pattern is provided. The method of relaxing hair or reducing curl pattern includes the steps of: administering a keratin protein composition that includes alpha keratin, gamma keratin, or a combination thereof, the administration resulting in keratin protein treated hair; administering to a surface of the keratin protein treated hair an activator composition including a co-initiator compound and a crosslinker compound to produce activator composition treated hair; and exposing the activator composition treated hair to a light source thereby causing the hair to relax and reduce curl pattern. According to one embodiment, the hair frizziness is reduced by at least 40%. According to one embodiment, the method of relaxing hair or reducing curl pattern includes the step of: adjusting one or more of: (i) light wavelength; (ii) light intensity; (iii) temperature; (iv) duration of light exposure; (v) amount and length of time the activator composition remains in the hair; (vi) weight percent of components included in the keratin protein composition; and (vii) weight percent of components included in the activator composition. According to one embodiment, the hair is exposed to the light source for at least one minute. According to one embodiment, the light source emits a light having a wavelength of from about 450 nm to about 550 nm.

### DESCRIPTION OF THE DRAWINGS

FIG. 1 is a photo of hair swatches showing the results of one photochemical composition treatment according to Example 1.

FIG. 2 is a photo of hair swatches showing the results after a washing and conditioning of the hair swatches of FIG. 1.

FIG. 3 is a photo of hair swatches showing the results of one photochemical composition treatment according to Example 2.

FIG. 4 is a photo of hair swatches showing the result after a washing and conditioning of the hair swatches of FIG. 3.

FIG. 5 is a photo of hair swatches showing the results of one photochemical composition treatment according to Example 3.

FIG. 6 is a photo of hair swatches showing the results after a washing and conditioning of the hair swatches of FIG. 5.

FIG. 7 is a photo of hair swatches showing the results of one photochemical composition treatment according to Example 4.

FIG. 8 is a photo of hair swatches showing the results after a washing and conditioning of the hair swatches of FIG. 7.

FIG. 9 is a photo of hair swatches showing the results of one photochemical composition treatment according to Example 5.

FIG. 10 is a photo of hair swatches showing the results after a washing and conditioning of the hair swatches of FIG. 9.

FIG. 11 is a photo of hair swatches showing the results of one photochemical composition treatment according to Example 6.

FIG. 12 is a photo of hair swatches showing the results after a washing and conditioning of the hair swatches of FIG. 11.

FIG. 13 is a photo of hair swatches showing the results of one photochemical composition treatment according to Example 7.

FIG. 14 is a photo of hair swatches showing the results after a washing and conditioning of the hair swatches of FIG. 13.

FIG. 15 is a photo of hair swatches showing the results of one photochemical composition treatment according to Example 8.

FIG. 16 is a photo of hair swatches showing the results after a washing and conditioning of the hair swatches of FIG. 15.

FIG. 17 is a photo of hair swatches showing the results of one photochemical composition treatment according to Example 9.

FIG. 18 is a photo of hair swatches showing the results after a washing and conditioning of the hair swatches of FIG. 17.

FIG. 19 is a photo of hair swatches showing the results of one photochemical composition treatment according to Example 10.

FIG. 20 is a photo of hair swatches showing the results after a washing and conditioning of the hair swatches of FIG. 19.

FIG. 21 is photo of hair swatches showing the effectiveness of L-arginine at 1.2% w/w, 10% w/w, 14% w/w and 20% w/w when using a hinged flatiron.

FIG. 22 is photo of hair swatches showing the effectiveness of L-arginine concentrations at 1.2% w/w, 10% w/w, 14% w/w and 20% w/w when using a wrap.

FIG. 23 is photo of hair swatches showing the effectiveness of keratin protein at concentrations of 2% w/w, 4% w/w, and 6% w/w (and no protein—water only) when using a flatiron.

### DETAILED DESCRIPTION

One or more aspects and embodiments may be incorporated in a different embodiment although not specifically described. That is, all aspects and embodiments can be combined in any way or combination. When referring to the compounds disclosed herein, the following terms have the following meanings unless indicated otherwise. The following definitions are meant to clarify, but not limit, the terms defined. If a particular term used herein is not specifically defined, such term should not be considered indefinite. Rather, terms are used within their accepted meanings.

As used herein, the term “keratin protein source” includes proteinaceous sources of keratin proteins from human hair. The human hair can be end-cut, as one would typically find in a barber shop or salon.

As used herein, the term “keratin protein(s)” as used herein collectively refers to hair protein sources, including but not limited to naturally occurring keratin, reduced keratin, oxidized keratin, S-sulfonated keratin, or a combination thereof. This term refers to the extracted keratin derivatives that are produced by oxidative treatment of keratin, including but not limited to keratin, alpha-keratin, gamma-keratin, or combinations thereof. Soluble keratins can be extracted from human hair fibers by oxidation using methods known in the art.

The term “alpha keratin” as used herein refers to a type of keratin protein. Alpha keratin includes the keratin protein that is a large, spring-shaped protein. Alpha keratin as provided herein nourishes or otherwise treats and improves the health of the scalp microbiome, repairs damage along the hair fiber, and reinforces hair strength. The alpha keratin may vary in molecular weight but typically falls in a range of from about 60 Da to about 6.2 MDa. The alpha keratin may be present as a whole, human keratin protein similar or substantially identical in protein conformation to native alpha keratin protein found in hair, skin and nails.

The term “gamma keratin” as used herein refers to a type of keratin protein that includes small, globular proteins that treat hair to provide elasticity and flexibility to hair. The gamma keratins provided herein also aid in the treatment and prevention of hair breakage (i.e., hair breakage reduction) and, when combined with alpha keratin, aids in the treatment and prevention of hair loss (e.g. hair fall-out). The gamma protein may maintain a protein conformation that is similar or identical to the native gamma keratin protein found in hair, skin and nails.

The term “molecular weight” as used herein refers to peak average molecular weight (Mp) which may be determined by methods known in the art such as through high performance liquid chromatography.

The terms “hair” and “mammalian hair” refer to hair on any part of the body of a mammal, and can include but is not limited to facial, cranial, or body hair and can further include hair on the scalp, head, neck, beard, moustache, eyebrows and sideburns.

The term “effective amount,” as used herein, means an amount of a composition sufficient to attain the intended result.

The term “activator” and “activation” may be used interchangeably and refer a photochemical reaction initiated by a composition's absorption of energy in the form of light.

The term “hair styling photochemical composition” refers to a composition including both the keratin protein composition and activator composition as provided herein.

The term “lux” as used herein refers to unit of measure of light level intensity in lumen per square meter. For example, one lux is the luminous flux of one lumen falling perpendicularly on a surface of one square meter.

### Overview

Current practices of changing the texture of hair require the use of chemicals that are harsh to both hair and scalp. These chemicals often result in high pH and high temperature that significantly damages hair in an effort to obtain the desired styling results. Further, these harsh chemical are coming under increased scrutiny from various legislative bodies. The present disclosure provides compositions and methods that improve hair styling results (i.e., achieve a desired result). In particular embodiments, wavelengths of light are applied to hair treated with a keratin protein composition and photochemical activator composition to obtain the desired styling results without the use of harsh chemicals or high temperatures that damage hair. According to a particular embodiment, the keratin protein composition and photochemical activator composition are combined prior to application to form a single hair styling photochemical composition.

The compositions and methods provided herein increase manageability of hair, reduces frizz, and may result in a curl, wave or relaxed hair depending on varying various factors including the total amount and length of time the activator composition is left in the hair, the intensity of light applied to the activator treated hair, the length of time of light exposure, the amount and duration of heat exposure, and weight percent of components included in the keratin protein composition and activator composition. The compositions and methods provided herein may be utilized on bleached, dyed or naturally colored hair.

### Keratin Protein Composition

A keratin protein composition is provided. The keratin protein composition may be applied to hair directly as provided herein or combined with the activator composition prior to application. The keratin protein composition repairs hair and protects the hair from future damage. The keratin protein composition also repairs current damage and equalizes the porosity of the hair prior to and during treatment. The keratin protein composition also helps to make the hair shaft a blank canvas for which the photochemical can work on to create the new desired texture including reduction of frizz and degree of curl.

According to one embodiment, the keratin protein composition includes alpha keratin, gamma keratin, or a combination thereof. According to one embodiment, the protein composition includes only alpha keratin (i.e., purely alpha keratin). According to one embodiment, the protein composition includes only gamma keratin (i.e., purely gamma keratin).

According to a particular embodiment, the keratin protein composition includes both alpha keratin and gamma keratin. According to one embodiment, the alpha keratin to gamma keratin ratio present in the protein composition is in a ratio found in native hair. According to one embodiment, the alpha keratin to gamma keratin ratio in the protein composition is in a ratio not found in native hair. According to one embodiment, the alpha keratin to gamma keratin is present in a ratio of about 1:1. According to one embodiment, the alpha keratin to gamma keratin is present in a ratio of about 2:1. According to one embodiment, the alpha keratin to gamma keratin is present in a ratio of about 1:2. According to one embodiment, the alpha keratin to gamma keratin is present in a ratio of about 3:1. According to one embodiment, the alpha keratin to gamma keratin is present in a ratio of about 1:3. According to one embodiment, the alpha keratin to gamma keratin is present in a ratio of about 4:1. According to one embodiment, the alpha keratin to gamma keratin is present in a ratio of about 1:4. According to one embodiment, the alpha keratin to gamma keratin is present in a ratio of about 5:1. According to one embodiment, the alpha keratin to gamma keratin is present in a ratio of about 1:5.

According to one embodiment, the keratin protein composition includes alpha keratin in an amount of at least about 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% by weight (w/w %) based on the total keratin in the keratin protein composition. According to one embodiment, the keratin protein composition includes gamma keratin in an amount of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95% or 99% by weight (w/w %) based on the total keratin in the keratin protein composition.

According to one embodiment, the keratin protein present (i.e., alpha keratin, gamma keratin, or a combination thereof) is present in the keratin protein composition in an amount of about 0.01% w/w to about 10% w/w based on the total weight of the keratin protein composition. According to one embodiment, the keratin protein is present in the keratin protein composition in an amount of about 1% w/w to about 9% w/w. According to one embodiment, the keratin protein is present in the keratin protein composition in an amount of about 2% w/w to about 8% w/w. According to one embodiment, the keratin protein is present in the keratin protein composition in an amount of about 3% w/w to about 7% w/w. According to one embodiment, the keratin protein is present in the keratin protein composition in an amount of about 4% w/w to about 6% w/w. According to one embodiment, the keratin protein is present in the keratin protein composition in an amount of about 6% w/w. According to one embodiment, the keratin protein is present in the keratin protein composition in an amount of up to about 6% w/w.

According to one embodiment, the keratin protein composition includes alpha keratin in an amount of at least about 90% w/w to about 99% w/w and gamma keratin in an amount of at least about 1% w/w to about 10% w/w based on the keratin in the keratin protein composition. According to one embodiment, the keratin protein composition includes alpha keratin in an amount of at least about 92% w/w to about 98% w/w and gamma keratin in an amount of at least about 2% w/w to about 8% w/w based on the keratin in the keratin protein composition. According to one embodiment, the keratin protein composition includes alpha keratin in an amount of at least about 93% w/w to about 97% w/w and gamma keratin in an amount of at least about 3% w/w to about 7% w/w based on the keratin in the keratin protein composition. According to one embodiment, the keratin protein composition includes alpha keratin in an amount of at least about 94% w/w to about 96% w/w and gamma keratin in an amount of at least about 4% w/w to about 6% w/w based on the keratin in the keratin protein composition. According to one embodiment, the keratin protein composition includes alpha keratin in an amount of at least about 95% w/w and gamma keratin in an amount of at least about 5% w/w based on the keratin in the keratin protein composition.

The alpha keratin may vary in peak average molecular weight (Mp) but typically falls in a range of from about 60 Da to about 6.2 MDa. According to one embodiment, the alpha keratin protein included in the keratin protein composition exhibits a peak average molecular weight (Mp) of at least about 443kDa. According to one embodiment, the alpha keratin of the keratin protein composition exhibits a peak average molecular weight (Mp) of at least about 500kDa. According to one embodiment, the alpha keratin of the keratin protein composition exhibits a peak average molecular weight (Mp) of at least about 600kDa. According to one embodiment, the alpha keratin of the keratin protein composition exhibits a peak average molecular weight (Mp) of at least about 700kDa. According to one embodiment, the alpha keratin of the keratin protein composition exhibits a peak average molecular weight (Mp) of at least about 800kDa. According to one embodiment, the alpha keratin of the keratin protein composition exhibits a peak average molecular weight (Mp) of at least about 850kDa. According to one embodiment, the alpha keratin of the keratin protein composition exhibits a peak average molecular weight (Mp) of at least about 900kDa. According to one embodiment, the alpha keratin of the keratin protein composition exhibits a peak average molecular weight (Mp) of at least about 1.0MDa. According to one embodiment, the alpha keratin of the keratin protein composition exhibits a peak average molecular weight (Mp) of at least about 1.1MDa. According to one embodiment, the alpha keratin of the keratin protein composition exhibits a peak average molecular weight (Mp) of at least about 1.2MDa. According to one embodiment, the alpha keratin of the keratin protein composition exhibits a peak average molecular weight (Mp) of at least about 1.3MDa.

According to one embodiment, at least about 30% of the alpha keratin of the keratin protein composition exhibits a peak average molecular weight (Mp) of at least about 443kDa. According to one embodiment, at least about 30% of the alpha keratin of the keratin protein composition exhibits a peak average molecular weight (Mp) of at least about 500kDa. According to one embodiment, at least about 30% of the alpha keratin of the keratin protein composition exhibits a peak average molecular weight (Mp) of at least about 600kDa. According to one embodiment, at least about 30% of the alpha keratin of the keratin protein composition exhibits a peak average molecular weight (Mp) of at least about 700kDa.

According to one embodiment, at least about 30% of the alpha keratin of the keratin protein composition exhibits a peak average molecular weight (Mp) of at least about 800kDa. According to one embodiment, at least about 30% of the alpha keratin of the keratin protein composition exhibits a peak average molecular weight (Mp) of at least about 850kDa. According to one embodiment, at least about 30% of the alpha keratin of the keratin protein composition exhibits a peak average molecular weight (Mp) of at least about 900kDa. According to one embodiment, at least about 30% of the alpha keratin of the keratin protein composition exhibits a peak average molecular weight (Mp) of at least about 1.0MDa. According to one embodiment, at least about 30% of the alpha keratin of the keratin protein composition exhibits a peak average molecular weight (Mp) of at least about 1.1MDa. According to one embodiment, at least about 30% of the alpha keratin of the keratin protein composition exhibits a peak average molecular weight (Mp) of at least about 1.2MDa. According to one embodiment, at least about 30% of the alpha keratin of the keratin protein composition exhibits a peak average molecular weight (Mp) of at least about 1.3MDa.

According to one embodiment, the gamma keratin included in the keratin protein composition exhibits a peak average molecular weight (Mp) of from about 15kDa to about 45kDa. According to one embodiment, the gamma keratin exhibits a peak average molecular weight (Mp) of from about 25kDa to about 35kDa. According to one embodiment, the gamma keratin exhibits a peak average molecular weight (Mp) of about 30kDa.

The alpha keratin and gamma keratin may be extracted according to the methods set forth in WO 2019/005724, the contents of which are incorporated herein by reference. The alpha keratin and gamma keratin are not degraded and maintain native protein conformation.

According to one embodiment, the keratin protein composition further includes ethylenediaminetetraacetic acid (EDTA). According to one embodiment, the EDTA is present in an amount of from about 0.01% w/w to about 3% w/w based on the total weight of the protein composition. According to one embodiment, the EDTA is present in an amount of from about 1% w/w to about 3% w/w based on the total weight of the protein composition. According to one embodiment, the EDTA is present in an amount of from about 1.5% w/w based on the total weight of the protein composition. According to one embodiment, the remainder of the protein composition includes water. The water may be purified according to a particular embodiment.

### Activator Composition

According to one embodiment, an activator composition is provided. The activator composition may be applied to hair directly as provided herein or combined with the keratin composition prior to application. Application of light to the activator composition initiates a photochemical reaction via absorption of light energy. According to a particular embodiment, the combination of light and activator composition initiates a reaction that helps change the hair texture by changing hair shape (curl pattern) and reducing hair frizz while not damaging the hair.

According to one embodiment, the activator composition includes at least one co-initiator compound and at least one crosslinker compound. According to one embodiment, the at least one co-initiator compound and at least one crosslinker compound are safe for use on hair without causing damage to the hair or scalp.

According to one embodiment, the co-initiator compound includes one or more persulfate compound. According to one embodiment, the co-initiator compound includes one or more of ammonium persulfate, potassium sulfate or sodium persulfate. According to one embodiment, the co-initiator compound includes one or more amino acid. According to one embodiment, the amino acid includes any essential or semi-essential amino acid. According to one embodiment, the amino acid includes one or more of L-arginine, valine, phenylalanine, leucine, isoleucine, threonine, tryptophan, histidine, and methionine. According to one embodiment, the co-initiator compound includes a persulfate compound, an amino acid, or a combination thereof.

According to one embodiment, the co-initiator compound is present in the activator composition in an amount of from about 0.01% w/w to about 30% w/w based on the total weight of the activator composition. According to one embodiment, the co-initiator compound is present in the activator composition in an amount of from about 5% w/w to about 29% w/w based on the total weight of the activator composition. According to one embodiment, the co-initiator compound is present in the activator composition in an amount of from about 10% w/w to about 30% w/w based on the total weight of the activator composition.

According to one embodiment, the crosslinker compound includes one or more photoinitiator compound. According to one embodiment, the crosslinker compound includes one or more type I or type II photoinitiator compound. According to one embodiment, the crosslinker compound includes one or more type II photoinitiator compound. According to a particular embodiment, the type II photoinitiator compound includes one or more B vitamins, coenzymes, or a combination thereof. According to a particular embodiment, the type II photoinitiator compound includes one or more of thiamin, riboflavin, niacin, pantothenic acid, pyridoxine, biotin, folate or folic acid, and cyanocobalamin. According to a particular embodiment, the type II photoinitiator compound includes riboflavin (riboflavin-5-phosphate).

According to one embodiment, the crosslinker compound is present in the activator composition in an amount of from about 0.01% w/w to about 10% w/w based on the total weight of the activator composition. According to one embodiment, the crosslinker compound is present in the activator composition in an amount of from about 1% w/w to about 9% w/w based on the total weight of the activator composition. According to one embodiment, the crosslinker compound is present in the activator composition in an amount of from about 2% w/w to about 8% w/w based on the total weight of the activator composition. According to one embodiment, the crosslinker compound is present in the activator composition in an amount of from about 3% w/w to about 7% w/w based on the total weight of the activator composition. According to one embodiment, the crosslinker compound is present in the activator composition in an amount of from about 4% w/w to about 6% w/w based on the total weight of the activator composition. According to one embodiment, the crosslinker compound is present in the activator composition in an amount of about 5% w/w based on the total weight of the activator composition.

According to one embodiment, the activator composition further includes a cosmetic base solution. The base solution may include one or more ingredients that do not impact or change the shape or conformation of the keratin proteins. Such ingredients may include one or more naturally occurring components. The base solution may include one or more of water, pink pomelo, hydrolyzed quinoa, artichoke leaf, vitamin A, vitamin C, vitamin B1, zinc, gotu kola, tapioca starch, kaolin clay, pea protein, phospholipids, brown algae, silica silylate, hydrated silica, asiaticoside/madecassoside, panthenol, cypress, baobab seed oil, or any combination thereof. The base solution may also include one or more of tocopherol, dimethicone, parabens, titanium dioxide, sodium lauryl sulphate, sodium laureth sulphate, retinol, collagen, ambergris, squalene, cochineal dye, guanine, tallow, gelatin, lanolin, citric acid, sodium citrate, cocamide, guar gum, xanthum gujm, glycol distearate, polyglycol esters, sodium chloride, glycerin, cetyl alcohol, stearyl alcohol, panthenol, silicones, or any combination thereof. The base solution may also include one or more of D-panthenol, polysorbate 20, cetyl alcohol, crambe abyssinica seed oil, hydroxyethyl acrylate/sodium acryloyldimethyl taurate copolymer, isohexadecane, polysorbate 60, cyclopentasiloxane, cyclohexasiloxane, 011-13 isoparaffin, dimethiconol, isohexadecane, dimethicone, caprylyl glycol, phenoxyethanol, hexylene glycol, or any combination thereof. The base solution may include one or more of the cosmetic additives, polymers, solvents, or film forming agents as described herein.

### Activation

A light source is utilized to activate the activator composition. According to one embodiment, the light source may emit a light at an intensity of at least 1,000 lux. According to one embodiment, the light source may emit a light at an intensity of at least 5,000 lux. According to one embodiment, the light source may emit a light at an intensity of at least 10,000 lux. According to one embodiment, the light source may emit a light at an intensity of at least 20,000 lux. According to one embodiment, the light source may emit a light at an intensity of at least 30,000 lux. According to one embodiment, the light source may emit a light at an intensity of at least 40,000 lux. According to one embodiment, the light source may emit a light at an intensity of at least 50,000 lux. According to one embodiment, the light source may emit a light at an intensity of at least 60,000 lux or more.

The light source may emit light at a wavelength at or within the visible light spectrum. The light source may emit light at a wavelength at or within the ultraviolet, blue, or red light spectrum. According to a particular embodiment, the light source emits a plurality of wavelengths at or within the ultraviolet, blue, or red light spectrum. According to a particular embodiment, the light source includes one or more light bulbs. Suitable light bulbs include, but are not limited to, those bulbs considered to be light emitting diodes (LED). According to a particular embodiment, the light source does not alter the hair's protein structure or break disulfide bonds/bridges.

According to a particular embodiment, the light source emits a light having a wavelength of below 150 nm. According to a particular embodiment, the light source emits a light having a wavelength of below 145 nm. According to a particular embodiment, the light source emits a light having a wavelength of below 140 nm. According to a particular embodiment, the light source emits a light having a wavelength of from about 1 nm to about 150 nm. According to a particular embodiment, the light source emits a light having a wavelength of from about 1 nm to about 145 nm.

According to a particular embodiment, the light source emits a light having a wavelength of from about 100 nm to about 400 nm. According to a particular embodiment, the light source emits a light having a wavelength of from about 150 nm to about 400 nm. According to a particular embodiment, the light source emits a light having a wavelength of from about 155 nm to about 400 nm. According to a particular embodiment, the light source emits a light having a wavelength of from about 100 nm to about 395 nm. According to a particular embodiment, the light source emits a light having a wavelength of from about 150 nm to about 350 nm. According to a particular embodiment, the light source emits a light having a wavelength of less than about 400 nm. According to a particular embodiment, the light source emits a light having a wavelength of less than about 395 nm.

According to a particular embodiment, the light source emits a light having a wavelength of at least 100 nm. According to a particular embodiment, the light source emits a light having a wavelength of at least 150 nm. According to a particular embodiment, the light source emits a light having a wavelength of at least 200 nm. According to a particular embodiment, the light source emits a light having a wavelength of at least 250 nm. According to a particular embodiment, the light source emits a light having a wavelength of at least 300 nm. According to a particular embodiment, the light source emits a light having a wavelength of at least 350 nm. According to a particular embodiment, the light source emits a light having a wavelength of at least 400 nm. According to a particular embodiment, the light source emits a light having a wavelength of at least 450 nm. According to a particular embodiment, the light source emits a light having a wavelength of at least about 500 nm.

According to a particular embodiment, the light source emits a light having a wavelength of less than 500 nm. According to a particular embodiment, the light source emits a light having a wavelength of less than 495 nm. According to a particular embodiment, the light source emits a light having a wavelength of from about 380 nm to about 495 nm. According to a particular embodiment, the light source emits a light having a wavelength of from about 400 nm to about 495 nm. According to a particular embodiment, the light source emits a light having a wavelength of from about 425 nm to about 495 nm. According to a particular embodiment, the light source emits a light having a wavelength of from about 455 nm to about 495 nm.

The light source may emit a light at a wavelength at or within the red light spectrum. According to a particular embodiment, the light source emits a light having a wavelength of from about 600 nm to about 750 nm.

The light source may be housed or lodged within a photochemical device. The photochemical device may be any acceptable device that may expose hair to a light source. Suitable photochemical devices include, but are not limited to, curlers, flat irons, light source sheets, LED fabrics, and light source wraps.

According to one embodiment, the photochemical device is configured to expose the hair to the light source that optionally provides heat to the surface of the hair without damaging the hair. According to one embodiment, the photochemical device is configured to expose the hair to the light source at a temperature of no more than 200° C. According to one embodiment, the photochemical device is configured to expose the hair to the light source at a temperature of no more than 175° C. According to one embodiment, the photochemical device is configured to expose the hair to the light source at a temperature of no more than 150° C. According to one embodiment, the photochemical device is configured to expose the hair to the light source at a temperature of no more than 125° C. According to one embodiment, the photochemical device is configured to expose the hair to the light source at a temperature of no more than 100° C. According to one embodiment, the photochemical device is configured to expose the hair to the light source at a temperature of no more than 95° C. According to one embodiment, the photochemical device is configured to expose the hair to the light source at a temperature of no more than 90° C. According to one embodiment, the photochemical device is configured to expose the hair to the light source at a temperature of no more than 85° C. According to one embodiment, the photochemical device is configured to expose the hair to the light source at a temperature of no more than 80° C. According to one embodiment, the photochemical device is configured to expose the hair to the light source at a temperature of at least 20° C. According to one embodiment, the photochemical device is configured to expose the hair to the light source at a temperature of at least 25° C. According to one embodiment, the photochemical device is configured to expose the hair to the light source at a temperature of at least 20° C. to about 200° C. According to one embodiment, the photochemical device is configured to expose the hair to the light source at a temperature of at least 20° C. to about 100° C. According to one embodiment, the photochemical device is configured to expose the hair to the light source at a temperature of at least 20° C. to about 95° C.

During use, hair treated with the activator composition may be exposed to the light source for varying amounts of time and at varying intensities to achieve desired results. According to one embodiment, the longer the hair is exposed to light, the more relaxed the hair becomes (i.e., less curl). Desired results may be achieved by exposing the hair for shorter amounts of times if a higher light intensity is utilized. Conversely, desired results may be achieved if lower intensity light is used but exposed for longer times.

According to one embodiment, the hair is exposed to the light source for at least about one minute. According to one embodiment, the hair is exposed to the light source for at least about two minutes. According to one embodiment, the hair is exposed to the light source for at least about three minutes. According to one embodiment, the hair is exposed to the light source for at least about four minutes. According to one embodiment, the hair is exposed to the light source for at least about five minutes. According to one embodiment, the hair is exposed to the light source for at least about five to ten minutes. According to one embodiment, the hair is exposed to the light source for at least about fifteen minutes. According to one embodiment, the hair is exposed to the light source for at least about twenty minutes. According to one embodiment, the hair is exposed to the light source for at least about twenty-five minutes. According to one embodiment, the hair is exposed to the light source for at least about thirty minutes. According to one embodiment, the hair is exposed to the light source for from about one minute to about thirty minutes.

### Formulations

According to one embodiment, the keratin protein composition and activator composition may each be formulated to be applied to any part of the body where a hair treatment application is acceptable. The keratin protein composition and activator composition, when combined to form a single hair styling photochemical composition, may be formulated in the same manner as the individual keratin protein composition and activator composition formulations provided herein.

According to one embodiment, the keratin protein composition and activator composition may be formulated to be applied to hair, scalp or a combination thereof. According to one embodiment, the keratin protein composition and activator composition may be formulated as an shampoo, conditioner, aqueous solution, powder, gel, mousse/foam, spray, dressing, lotion, wax, hydrogel, oil, emulsion, paste, polish or cream. According to one embodiment, the keratin protein composition and activator composition as provided herein may be formulated as hair care products such as shampoo, conditioner, spray, or foam. According to one embodiment, the keratin protein composition and activator composition as provided herein may be formulated as or incorporated within a moisturizer, a deodorant, an anti-aging/skin repair preparation, a cleanser, a toner, an eye care composition, a lip care composition, a sun care composition (e.g., sunscreen), a hand care composition, or a body care composition.

### Methods of Treatment and Use

The keratin protein composition and activator composition (or hair styling photochemical composition when keratin protein composition and activator composition are combined) provided herein are suitable for various hair treatment purposes on the exterior of a mammalian body. The keratin protein composition and activator composition as provided herein may be utilized to strengthen or improve the overall appearance and health of a mammal's hair including reducing frizz and attaining a desired level of curl. The keratin protein composition and activator composition provided herein may be utilized in the manufacture of a medicament for the various uses or methods of treatment provided herein.

The keratin protein composition and activator composition provided herein may be topically administered to the hair (and/or scalp) for a period of time sufficient to bring about the desired level of improvement in the hair texture (e.g., reduction of frizz and degree of curl). A user may topically administer any of the compositions provided herein directly to the hair by gently massaging, spraying, dabbing, swabbing, or rubbing the requisite composition into the desired area. According to one embodiment, the keratin protein composition and activator composition provided herein may be topically administered to the hair in a single treatment in an effective amount and left in the hair for a period of time sufficient to bring about the desired level hair texture. Duration of treatment with the compositions may vary depending on the desired effect. The keratin protein composition and activator composition may be administered to the hair again via a single treatment at a subsequent point in time such as within one month, two months, three months, four months, five months, six months or more. In some embodiments, the degree of desired impact on the hair will vary directly with the total amount and length of time the activator composition is left in the hair, the intensity of light applied to the activator treated hair, the length of time of light exposure, the amount and duration of heat exposure, and weight percent of components included in the keratin protein composition and activator composition.

According to one aspect, a method of photochemical hair treatment is provided. The method includes the step of administering to a surface of the hair in need of treatment the keratin protein composition as provided herein. The hair in need of treatment may be wetted prior to administration of the protein composition. After administration of the protein composition, the hair may be combed one or more times. The protein composition may remain in the hair for at least one minute. According to one embodiment, the protein composition may remain in the hair for at least five minutes. According to one embodiment, the protein composition may remain in the hair for at least ten minutes. According to one embodiment, the protein composition may remain in the hair for at least fifteen minutes or any of the durations provided herein.

The method of photochemical hair treatment further includes the step of administering to a surface of the protein treated hair an activator composition that includes a co-initiator compound and a crosslinker compound as provided herein. The step of administering the activator composition to the protein treated hair may include messaging or rubbing down the length of the hair to provide activator treated hair. Alternatively, the step of administering the activator composition to the protein treated hair may include spraying the activator composition on the hair (via a spray bottle) or brushing the activator composition on the hair via a suitable instrument such as a color brush.

The method of photochemical hair treatment further includes the step of exposing the activator treated hair to a light source and, optionally, heat. According to one embodiment, the activator treated hair is exposed to the light source for at least about one minute. According to one embodiment, the activator treated hair is exposed to the light source for at least about two minutes. According to one embodiment, the activator treated hair is exposed to the light source for at least about three minutes. According to one embodiment, the activator treated hair is exposed to the light source for at least about four minutes. According to one embodiment, the activator treated hair is exposed to the light source for at least about five minutes. According to one embodiment, the activator treated hair is exposed to the light source for at least about five to ten minutes. According to one embodiment, the activator treated hair is exposed to the light source for at least about fifteen minutes. According to one embodiment, the activator treated hair is exposed to the light source for at least about twenty minutes. According to one embodiment, the activator treated hair is exposed to the light source for at least about twenty-five minutes. According to one embodiment, the activator treated hair is exposed to the light source for at least about thirty minutes. According to one embodiment, the activator treated hair is exposed to the light source for from about one minute to about thirty minutes.

The method of photochemical hair treatment may further include the step of removing the hair from the light source and rinsing the hair under lukewarm water to remove the applied compositions. The hair may then be washed, rinsed, and conditioned. After conditioning, the hair may be rinsed with lukewarm water and combed at least once. The hair may then be dried according to normal, acceptable procedures.

The method of photochemical hair treatment may further include the step of adjusting light wavelength, light intensity, temperature, duration of light exposure, amount and length of time the activator composition is left in the hair, weight percent of components included in the keratin protein composition and activator composition, or any combination thereof to achieve the desired hair curl pattern.

According to another aspect, a method of reducing hair frizziness is provided. The method includes the step of administering to a surface of the hair in need of treatment a keratin protein composition as provided herein. The method of reducing hair frizziness further includes the step of administering to a surface of the protein treated hair an activator composition as provided herein. The method of reducing hair frizziness further includes the step of exposing the activator treated hair to a light source and, optionally, heat, thereby causing the hair to exhibit less frizz. According to one embodiment, the hair frizziness is reduced by at least 10%, 20%, 30%, 40%, 50% or more. According to one embodiment, the hair is exposed to the light source for at least one minute or any duration provided herein. According to one embodiment, the light source emits a light having a wavelength of from about 1 nm to about 750 nm. The amount of desired frizz reduction may be achieved by the step of adjusting light wavelength, light intensity, temperature, duration of light exposure, amount and length of time the activator composition is left in the hair, weight percent of components included in the keratin protein composition and activator composition, or any combination thereof.

According to another aspect, a method of relaxing hair is provided. By relaxing hair, heavily curled hair may be straightened. The method of relaxing hair includes the step of administering to a surface of the hair in need of treatment a keratin protein composition as provided herein. The method of relaxing further includes the step of administering to a surface of the protein treated hair an activator composition as provided herein. The method of relaxing hair further includes the step of exposing the activator treated hair to a light source and, optionally, heat, thereby causing the hair to exhibit less curl. According to one embodiment, the hair frizziness is reduced by at least 10%, 20%, 30%, 40%, 50% or more. According to one embodiment, the hair is exposed to the light source for at least one minute or any duration provided herein. According to one embodiment, the light source emits a light having a wavelength of from about 1 nm to about 750 nm. The amount of desired relaxation may be achieved by the step of adjusting the total amount and length of time the activator composition is left in the hair, the intensity of light applied to the activator treated hair, the length of time of light exposure, the amount and duration of heat exposure, and weight percent of components included in the keratin protein composition and activator composition.

According to another aspect, a hair styling kit is provided. The hair styling kit includes a protein composition as well as an activator composition comprising as provided herein. According to one embodiment, the kit optionally includes a photochemical device, the photochemical device including a light source as provided herein. According to one embodiment, the kit may include a container such as a box or bag that includes the protein composition and activator composition that are, in turn, packaged separately with the container. According to one embodiment, the kit may include a set of instructions for use.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

### EXAMPLES

### Protein Composition

A protein composition was prepared and utilized in the Examples provided below. Except as where indicated, the protein composition was prepared according to the methods provided herein and included about 6% w/w of a native blend of water soluble proteins. Specifically, the proteins present included alpha and gamma oxidized keratin proteins present in ratios and amounts similar or substantially the same as the natural alpha and gamma proteins in hair. The protein composition further included an EDTA solution at an amount of about 1.5% w/w. The EDTA solution pH was adjusted to about 8.0 with NaOH for solubility. The remainder of the protein composition was purified water.

### Activator Composition—Sodium Persulfate Co-initiator

An activator composition was prepared and utilized in the Examples provided below. In some examples (Examples 1-5), the activator composition included riboflavin-5-phosphate in an amount of about 5% w/w and sodium persulfate in an amount of about 20% w/w as a co-initiator. The remainder of the composition was a suitable cosmetic base found in shampoos or conditioners.

### Activator Composition—L-Arginine Co-initiator

An activator composition was prepared and utilized in the Examples provided below. In some examples, the activator composition included riboflavin-5-phosphate in an amount of about 5% w/w and L-Arginine in an amount of about 14% w/w as a co-initiator. The remainder of the composition was water.

### Treatment Protocol

A wet coarse curly/frizzy hair swatch (about 2 grams) was placed under running lukewarm water for about one minute. Next, about one milliliter of the protein composition was applied to the hair swatch and combed through 5 times. The protein composition was allowed to sit on the hair for about 15 minutes after combing. About two milliliters of the activator composition was then applied to the hair swatch while rubbing the activator composition down the length of the hair swatch. The swatch was laid on the particular photochemical device while protecting the light source within the device with saran wrap. About two milliliters of activator composition was then applied along length of hair on light and rubbed in while flattening the swatch. The photochemical device was then turned on to the highest intensity setting for the particular device. The hair swatch was allowed to sit and process in the photochemical device for 1, 5, 10, 15, and 30 minutes. The hair swatch was removed from the photochemical device and rinsed under lukewarm water for about 30 seconds while rubbing the hair. Next, about 0.2 milliliters of shampoo was applied and massaged in the hair swatch for about 30 seconds. The hair swatch was rinsed under lukewarm water for 30 seconds while rubbing the hair. Next, about 0.2 milliliters of conditioner was applied and massaged in the hair swatch for about 30 seconds. The hair swatch was then rinsed under lukewarm water for 30 seconds while rubbing the hair and combing through about five times. The hair swatch was then gently shaken to assess any curl pattern. The hair swatch was then allowed to hang dry in a 37° C. oven until fully dry (at least about 4 hours).

### Example 1

### Two-Piece Flatiron (no faceplate)

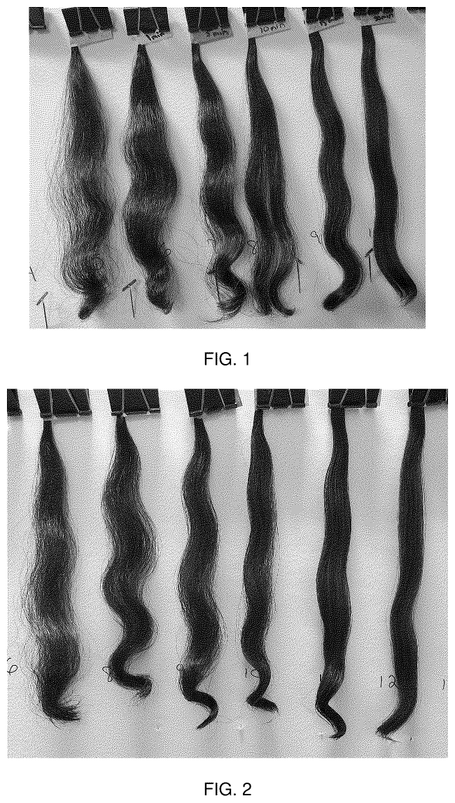

Testing was conducted on hair swatches according to the treatment protocol with an activator composition utilizing riboflavin-5-phosphate in an amount of about 5% w/w, sodium persulfate co-initiator in an amount of about 20% w/w and a two-piece flatiron missing a faceplate as the photochemical device. The wavelength utilized in the flatiron was from about 448 nm to about 454 nm. The max temperature reached was about 80° C. The average high intensity reached was about 42439 lux. A control was prepared utilizing water in place of the protein composition and activator composition. In FIG. 1, the first hair swatch on the left represents the control (water only). The second hair swatch through sixth hair swatch (left to right) represents photochemical device exposure times of 1 minute, 5 minutes, 10 minutes, 15 minutes, and 30 minutes, respectively. As evidenced in FIG. 1, the hair swatches showed increasingly manageable, relaxed, slightly curled hair with little to no frizz as the length of time of photochemical device exposure increased.

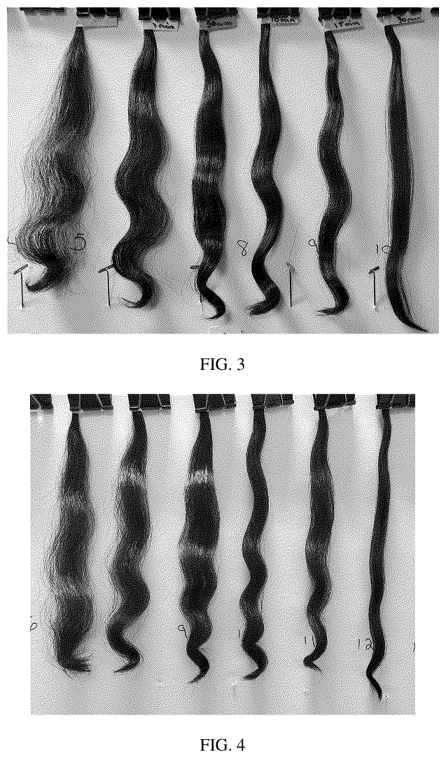

Each hair swatch of FIG. 1 was then washed, rinsed, conditioned and then rinsed once more with a traditional shampoo and conditioner available from Suave®. The results after the wash and condition are shown in FIG. 2.

### Example 2

### Hinged Flatiron

Testing was conducted on hair swatches according to the treatment protocol with an activator composition utilizing riboflavin-5-phosphate in an amount of about 5% w/w, sodium persulfate co-initiator in an amount of about 20% w/w and a hinged flatiron as the photochemical device. The wavelength utilized in the flatiron was from about 448 nm to about 453 nm. The max temperature reached was about 63° C. The average high intensity reached was about 33343 lux. A control was prepared utilizing water in place of the protein composition and activator composition. In FIG. 3, the first hair swatch on the left represents the control (water only). The second hair swatch through sixth hair swatch (left to right) represents photochemical device exposure times of 1 minute, 5 minutes, 10 minutes, 15 minutes, and 30 minutes, respectively. As evidenced in FIG. 3, the hair swatches showed increasingly manageable, relaxed, slightly curled hair with little to no frizz as the length of time of photochemical device exposure increased.

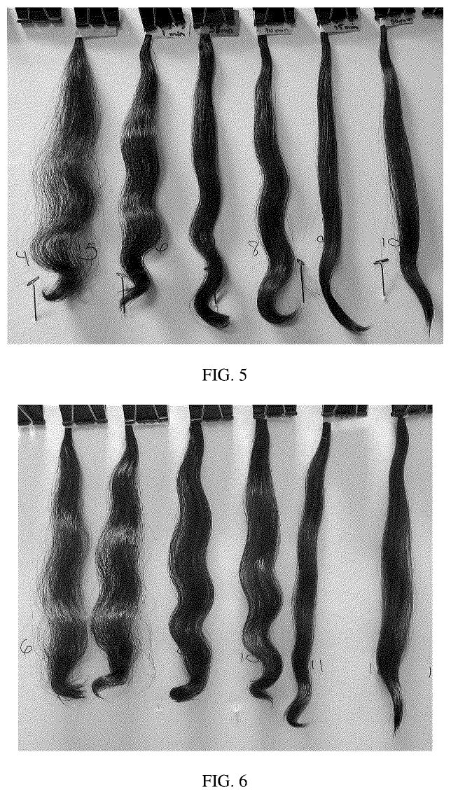

Each hair swatch was then washed, rinsed, conditioned and then rinsed once more with a traditional shampoo and conditioner available from Suave®. The results after the wash and condition are shown in FIG. 4.

### Example 3

### Hinged Flatiron

Testing was conducted on hair swatches according to the treatment protocol with an activator composition utilizing riboflavin-5-phosphate in an amount of about 5% w/w, sodium persulfate co-initiator in an amount of about 20% w/w and a hinged flatiron as the photochemical device. The wavelength utilized in the flatiron was from about 447 nm to about 453 nm. The max temperature reached was about 68° C. The average high intensity reached was about 39771 lux. A control was prepared utilizing water in place of the protein composition and activator composition. In FIG. 5, the first hair swatch on the left represents the control (water only). The second hair swatch through sixth hair swatch (left to right) represents photochemical device exposure times of 1 minute, 5 minutes, 10 minutes, 15 minutes, and 30 minutes, respectively. As evidenced in FIG. 5, the hair swatches showed increasingly manageable, relaxed, slightly curled hair with little to no frizz as the length of time of photochemical device exposure increased.

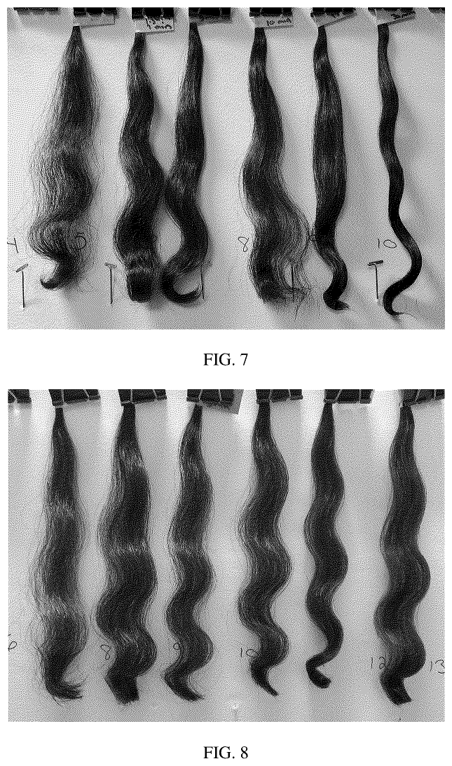

Each hair swatch was then washed, rinsed, conditioned and then rinsed once more with a traditional shampoo and conditioner available from Suave®. The results after the wash and condition are shown in FIG. 6.

### Example 4

### Wrap

Testing was conducted on hair swatches according to the treatment protocol with an activator composition riboflavin-5-phosphate in an amount of about 5% w/w, sodium persulfate co-initiator in an amount of about 20% w/w and a photochemical wrap device (i.e., flat towel-like device having a plurality of light sources to wrap the hair swatches). The wavelength utilized in the flatiron was from about 447 nm to about 454 nm. The max temperature reached was about 29° C. The average high intensity reached was about 29043 lux. A control was prepared utilizing water in place of the protein composition and activator composition. In FIG. 7, the first hair swatch on the left represents the control (water only). The second hair swatch through sixth hair swatch (left to right) represents photochemical device exposure times of 1 minute, 5 minutes, 10 minutes, 15 minutes, and 30 minutes, respectively. As evidenced in FIG. 7, the hair swatches showed increasingly manageable, relaxed, slightly curled hair with little to no frizz as the length of time of photochemical device exposure increased.

Each hair swatch was then washed, rinsed, conditioned and then rinsed once more with a traditional shampoo and conditioner available from Suave®. The results after the wash and condition are shown in FIG. 8.

### Example 5

### Curler Tape

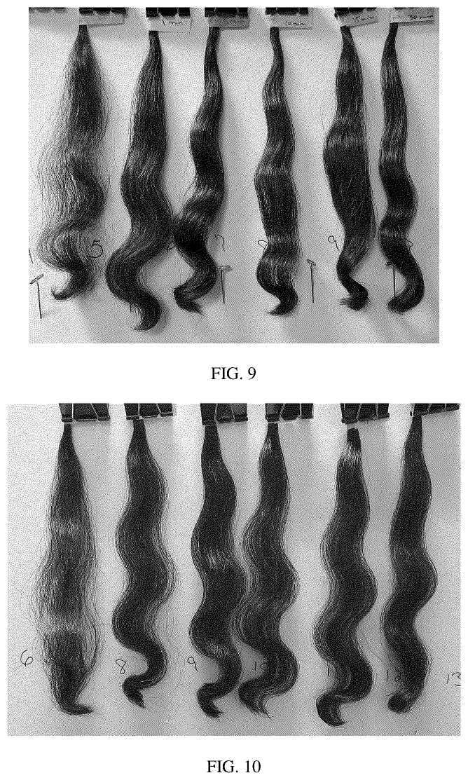

Testing was conducted on hair swatches according to the treatment protocol with an activator composition utilizing riboflavin-5-phosphate in an amount of about 5% w/w, sodium persulfate co-initiator in an amount of about 20% w/w and a photochemical curler tape (i.e., tape-like device having a plurality of light sources that wraps the hair swatches). The wavelength utilized in the flatiron was from about 447 nm to about 454 nm. The max temperature reached was about 29° C. The average high intensity reached was about 29043 lux. A control was prepared utilizing water in place of the protein composition and activator composition. In FIG. 9, the first hair swatch on the left represents the control (water only). The second hair swatch through sixth hair swatch (left to right) represents photochemical device exposure times of 1 minute, 5 minutes, 10 minutes, 15 minutes, and 30 minutes, respectively. As evidenced in FIG. 9, the hair swatches showed increasingly manageable, relaxed, slightly curled hair with little to no frizz as the length of time of photochemical device exposure increased.

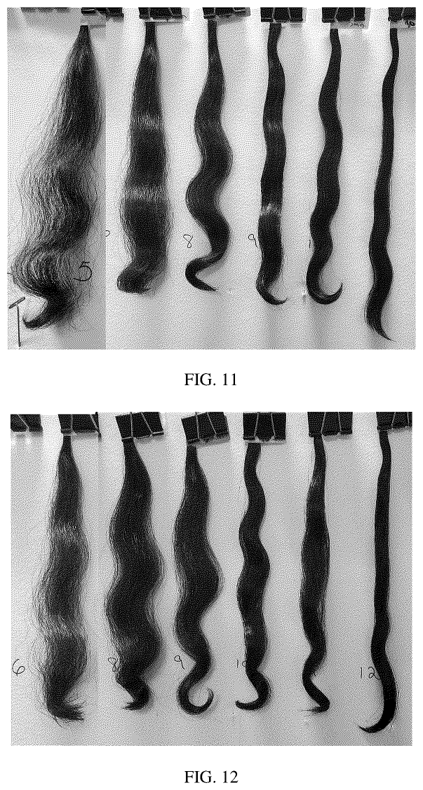

Each hair swatch was then washed, rinsed, conditioned and then rinsed once more with a traditional shampoo and conditioner available from Suave®. The results after the wash and condition are shown in FIG. 10.

In summary, Examples 1-5 demonstrated that the shorter light exposure resulted in hair that was considerably less frizzy compared to the control with hair curl pattern remaining intact. The longer length of time of light exposure resulted in relaxing of the hair and loss of curl pattern. The intermediate light exposures gave varying curl pattern between the long and short time of light exposures. All light sources presented viable options demonstrating that hair styling results can be controlled by varying light exposure duration.

### Example 6

### Two-Piece Flatiron (no faceplate)

Testing was conducted on hair swatches according to the treatment protocol with an activator composition utilizing riboflavin-5-phosphate in an amount of about 5% w/w, L-Arginine in an amount of about 14% w/w and a two-piece flatiron missing a faceplate as the photochemical device. The wavelength utilized in the flatiron was from about 448 nm to about 454 nm. The max temperature reached was about 80° C. The average high intensity reached was about 42439 lux. A control was prepared utilizing water in place of the protein composition and activator composition. In FIG. 11, the first hair swatch on the left represents the control (water only). The second hair swatch through sixth hair swatch (left to right) represents photochemical device exposure times of 1 minute, 5 minutes, 10 minutes, 15 minutes, and 30 minutes, respectively. As evidenced in FIG. 11, the hair swatches showed increasingly manageable, relaxed, slightly curled hair with little to no frizz as the length of time of photochemical device exposure increased.

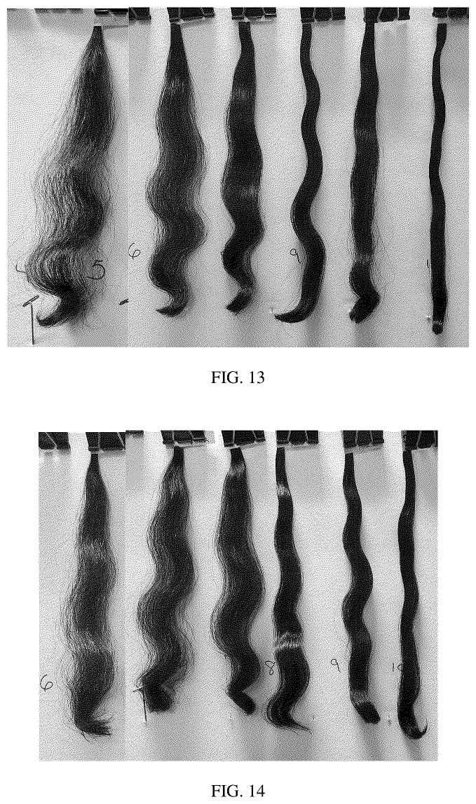

Each hair swatch was then washed, rinsed, conditioned and then rinsed once more with a traditional shampoo and conditioner produce available from Suave®. The results after the wash and condition are shown in FIG. 12.

### Example 7

### Hinged Flatiron

Testing was conducted on hair swatches according to the treatment protocol with an activator composition utilizing riboflavin-5-phosphate in an amount of about 5% w/w, L-Arginine in an amount of about 14% w/w and a hinged flatiron as the photochemical device. The wavelength utilized in the flatiron was from about 448 nm to about 453 nm. The max temperature reached was about 63° C. The average high intensity reached was about 33343 lux. A control was prepared utilizing water in place of the protein composition and activator composition. In FIG. 13, the first hair swatch on the left represents the control (water only). The second hair swatch through sixth hair swatch (left to right) represents photochemical device exposure times of 1 minute, 5 minutes, 10 minutes, 15 minutes, and 30 minutes, respectively. As evidenced in FIG. 13, the hair swatches showed increasingly manageable, relaxed, slightly curled hair with little to no frizz as the length of time of photochemical device exposure increased.

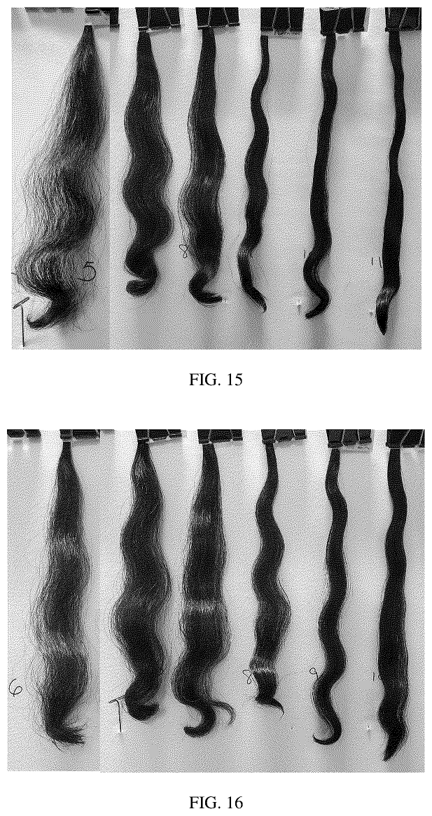

Each hair swatch was then washed, rinsed, conditioned and then rinsed once more with a traditional shampoo and conditioner available from Suave®. The results after the wash and condition are shown in FIG. 14.

### Example 8

### Hinged Flatiron

Further testing was conducted on hair swatches according to the treatment protocol with an activator composition utilizing riboflavin-5-phosphate in an amount of about 5% w/w, L-Arginine in an amount of about 14% w/w and a hinged flatiron as the photochemical device. The wavelength utilized in the flatiron was from about 447 nm to about 453 nm. The max temperature reached was about 68° C. The average high intensity reached was about 39771 lux. A control was prepared utilizing water in place of the protein composition and activator composition. In FIG. 15, the first hair swatch on the left represents the control (water only). The second hair swatch through sixth hair swatch (left to right) represents photochemical device exposure times of 1 minute, 5 minutes, 10 minutes, 15 minutes, and 30 minutes, respectively. As evidenced in FIG. 15, the hair swatches showed increasingly manageable, relaxed, slightly curled hair with little to no frizz as the length of time of photochemical device exposure increased.

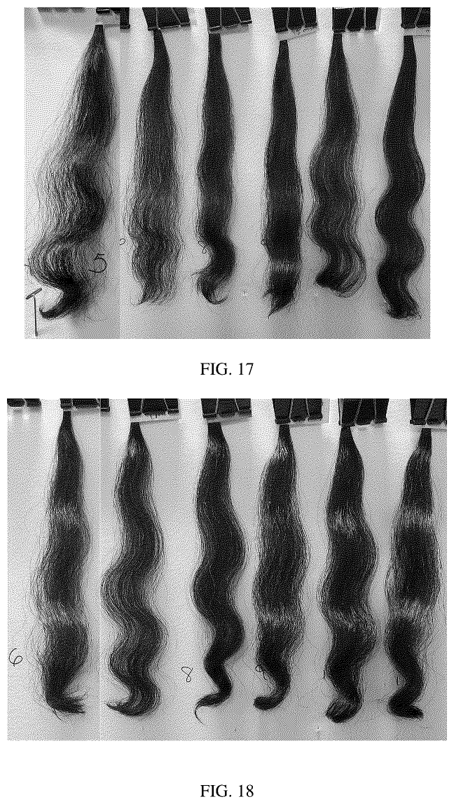

Each hair swatch was then washed, rinsed, conditioned and then rinsed once more with a traditional shampoo and conditioner available from Suave®. The results after the wash and condition are shown in FIG. 16.

### Example 9

### Wrap

Testing was conducted on hair swatches according to the treatment protocol with an activator composition utilizing riboflavin-5-phosphate in an amount of about 5% w/w, L-Arginine in an amount of about 14% w/w and a photochemical wrap device (i.e., flat towel-like device having a plurality of light sources to wrap the hair swatches). The wavelength utilized in the flatiron was from about 447 nm to about 454 nm. The max temperature reached was about 29° C. The average high intensity reached was about 29043 lux. A control was prepared utilizing water in place of the protein composition and activator composition. In FIG. 17, the first hair swatch on the left represents the control (water only). The second hair swatch through sixth hair swatch (left to right) represents photochemical device exposure times of 1 minute, 5 minutes, 10 minutes, 15 minutes, and 30 minutes, respectively. As evidenced in FIG. 17, the hair swatches showed increasingly manageable, relaxed, slightly curled hair with little to no frizz as the length of time of photochemical device exposure increased.

Each hair swatch was then washed, rinsed, conditioned and then rinsed once more with a traditional shampoo and conditioner available from Suave®. The results after the wash and condition are shown in FIG. 18.

### Example 10

### Curler Tape

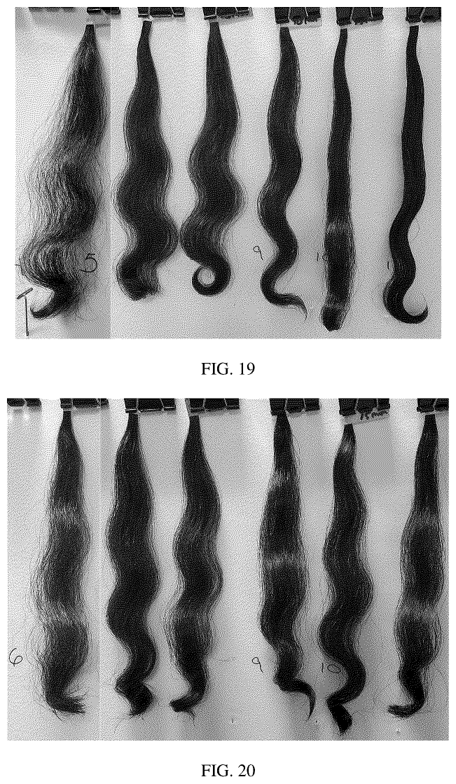

Testing was conducted on hair swatches according to the treatment protocol with an activator composition utilizing riboflavin-5-phosphate in an amount of about 5% w/w, L-Arginine in an amount of about 14% w/w and a photochemical curler tape (i.e., tape-like device having a plurality of light sources that wraps the hair swatches). The wavelength utilized in the flatiron was from about 447 nm to about 454 nm. The max temperature reached was about 29° C. The average high intensity reached was about 29043 lux. A control was prepared utilizing water in place of the protein composition and activator composition. In FIG. 19, the first hair swatch on the left represents the control (water only). The second hair swatch through sixth hair swatch (left to right) represents photochemical device exposure times of 1 minute, 5 minutes, 10 minutes, 15 minutes, and 30 minutes, respectively. As evidenced in FIG. 19, the hair swatches showed increasingly manageable, relaxed, slightly curled hair with little to no frizz as the length of time of photochemical device exposure increased.

Each hair swatch was then washed, rinsed, conditioned and then rinsed once more with a traditional shampoo and conditioner available from Suave®. The results after the wash and condition are shown in FIG. 20.

In summary, Examples 6-10 once again demonstrated that the shorter light exposure resulted in hair that was considerably less frizzy compared to the control, however, hair curl pattern remained intact. The longer light exposure resulted in loss of curl pattern. The intermediate light exposures gave varying between the long and short light exposures. All light sources presented viable options demonstrating that hair styling results can be controlled by varying light exposures. Examples 1-10 further demonstrated that L-arginine and sodium persulfate are both viable co-initiator compounds

### Example 11

### Varying L-Arginine

### Hinged Flatiron

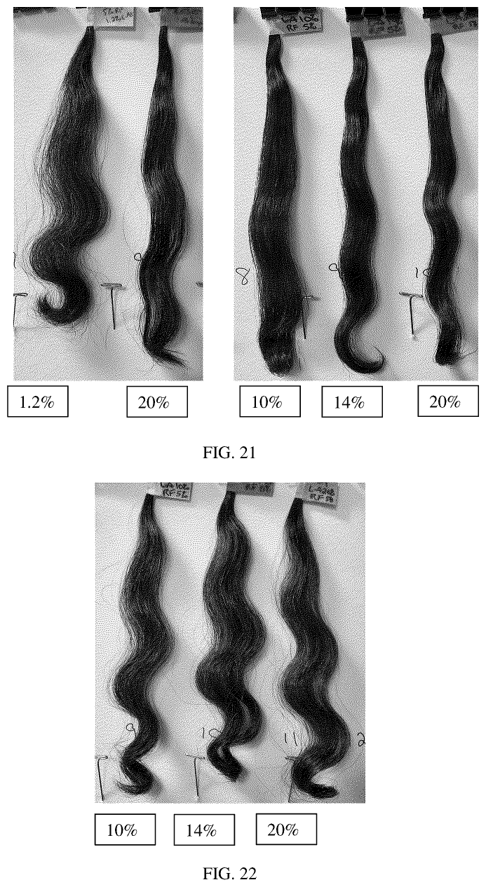

Testing was conducted on hair swatches according to the treatment protocol with an activator composition utilizing L-arginine co-initiator at varying concentrations and a two-piece flatiron missing a faceplate as the photochemical device. The wavelength utilized in the flatiron was from about 448 nm to about 454 nm. The max temperature reached was about 80° C. The average high intensity reached was about 42439 lux. The varying amounts of L-arginine applied to the hair swatches included 1.2% w/w, 10% w/w, 14% w/w and 20% w/w. The effectiveness of L-arginine was shown with positive visible results at all concentrations as evidenced in FIG. 21. Particularly, the higher the concentration of L-Arginine, the greater styling results attained.

### Example 12

### Varying L-Arginine

### Wrap

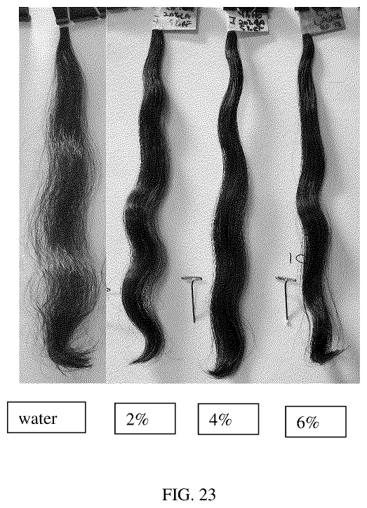

Testing was conducted on hair swatches according to the treatment protocol with an activator composition utilizing L-arginine co-initiator at varying concentrations and a wrap photochemical device. The wavelength utilized in the flatiron was from about 447 nm to about 454 nm. The max temperature reached was about 29° C. The average high intensity reached was about 29043 lux. The varying amounts of L-arginine applied to the hair swatches included 10% w/w, 14% w/w and 20% w/w. The effectiveness of L-arginine was shown with positive visible results at all concentrations as evidenced in FIG. 22. Particularly, the higher the concentration of L-Arginine, the greater styling results attained.

Examples 11-12 vary the L-arginine initiator amounts. The results demonstrate that hair texture may be changed (e.g., frizz reduction to outright curl pattern reduction) by altering the initiator content. The lower the concentration of L-arginine, the less frizz reduction and less change to curl pattern. The higher concentration of L-arginine, the higher frizz reduction and a larger change to the curl pattern (i.e., more relaxed).

### Example 13

### Varying Protein Concentration

### Hinged Flatiron

Testing was conducted on hair swatches according to the treatment protocol with an activator composition utilizing L-arginine co-initiator and the protein at varying concentrations. A two-piece flatiron missing a faceplate was used as the photochemical device. The wavelength utilized in the flatiron was from about 448 nm to about 454 nm. The max temperature reached was about 80° C. The average high intensity reached was about 42439 lux. The treatment was repeated for varying amounts of keratin protein including 2% w/w, 4% w/w, and 6% w/w (and no protein—water only). The effectiveness of the protein concentration compared to just water was shown with positive visible results at all concentrations as evidenced in FIG. 23.

### General Statements

The following statements provide a general description of the disclosure and are not intended to limit the appended claims.

Statement 1. A hair styling kit is provided that includes:
a protein composition including alpha keratin, gamma keratin, or a combination thereof; and
an activator composition including a co-initiator compound and a crosslinker compound.

Statement 2. The hair styling kit according to Statement 1, further including a photochemical device, the photochemical device including a light source.

Statement 3. The hair styling kit according to Statements 1-2, wherein the protein composition further comprises ethylenediaminetetraacetic acid (EDTA).

Statement 4. The hair styling kit according to Statements 1-3, wherein the EDTA is present in an amount of from about 0.01% w/w to about 3% w/w based on the total weight of the protein composition.

Statement 5. The hair styling kit according to Statements 1-4, wherein the protein composition further comprises water.

Statement 6. The hair styling kit according to Statements 1-5, wherein the activator composition further comprises a cosmetic base solution.

Statement 7. The hair styling kit according to Statements 1-6, wherein the co-initiator compound is sodium persulfate, L-arginine, or a combination thereof.

Statement 8. The hair styling kit according to Statements 1-7, wherein the crosslinker compound is riboflavin-5-phosphate.

Statement 9. The hair styling kit according to Statements 1-8, wherein the alpha keratin, gamma keratin, or combination thereof are present in the protein composition in an amount of about 0.01% w/w to about 10% w/w based on the total weight of the protein composition.

Statement 10. The hair styling kit according to Statements 1-9, wherein the crosslinker compound is present in the activator composition in an amount of from about 0.01% w/w to about 10% w/w based on the total weight of the activator composition.

Statement 11. The hair styling kit according to Statements 1-10, wherein the co-initiator compound is present in the activator composition in an amount of from about 0.01% w/w to about 30% w/w based on the total weight of the activator composition.

Statement 12. The hair styling kit according to Statements 1-11, wherein the keratin protein composition includes both alpha keratin and gamma keratin.

Statement 13. The hair styling kit according to Statements 1-12, wherein the alpha keratin to gamma keratin is present in the keratin protein composition at a ratio found in native hair.

Statement 14. The hair styling kit according to Statements 1-13, wherein the alpha keratin to gamma keratin is present in the keratin protein composition at a ratio not found in native hair.

Statement 15. The hair styling kit according to Statements 1-14, wherein the alpha keratin to gamma keratin is present in the keratin protein composition at a ratio of from about 2:1 to about 4:1.

Statement 16. The hair styling kit according to Statements 1-15, wherein the total keratin protein content of the keratin protein composition is at least 2% w/w based on the total weight of the keratin protein composition.

Statement 17. The hair styling kit according to Statements 1-16, wherein the total keratin protein content of the keratin protein composition is at least 4% w/w based on the total weight of the keratin protein composition.

Statement 18. The hair styling kit according to Statements 1-17, wherein the total keratin protein content of the keratin protein composition is at least 6% w/w based on the total weight of the keratin protein composition.

Statement 19. The hair styling kit according to Statements 1-18, wherein the total keratin protein content of the keratin protein composition is between about 2% w/w to about 6% w/w based on the total weight of the keratin protein composition.

Statement 20. A two-part hair styling composition is provided that includes a keratin protein composition comprising alpha keratin, gamma keratin, or a combination thereof as provided in statements 1-19; and an activator composition comprising a co-initiator compound and a crosslinker compound as provided in statements 1-19, wherein upon exposure to light, the photochemical composition is activated.

Statement 21. A method of photochemical hair treatment is provided that includes the steps of:
administering to a surface of the hair in need of treatment a protein composition as provided in statements 1-19, the administration resulting in protein treated hair;
administering to a surface of the protein treated hair an activator composition comprising a co-initiator compound and a crosslinker compound as provided in statements 1-19 resulting in activator composition treated hair; and
exposing the hair treated with the activator composition to a light source, and, optionally exposing the hair treated with the activator composition to heat.

Statement 22. The method of statement 21, wherein the activator composition treated hair is exposed to the light source for at least one minute.

Statement 23. The method of statements 21-22, wherein the light source emits a light having a wavelength of from about 1 nm to about 750 nm.

Statement 24. The method of statements 21-23, wherein the alpha keratin, gamma keratin, or combination thereof are present in the protein composition in an amount of about 0.01% w/w to about 10% w/w based on the total weight of the protein composition.

Statement 25. The method of statements 21-24, wherein the crosslinker compound is riboflavin-5-phosphate.

Statement 26. The method of statements 21-25, wherein the crosslinker compound is present in the activator composition in an amount of from about 0.01% w/w to about 10% w/w based on the total weight of the activator composition.

Statement 27. The method of statements 21-26, wherein the co-initiator compound is sodium persulfate, L-arginine, or a combination thereof.

Statement 28. The method of statements 21-27, wherein the co-initiator compound is present in the activator composition in an amount of from about 0.01% w/w to about 30% w/w based on the total weight of the activator composition.

Statement 29. A method of reducing hair frizziness comprising the steps of:
administering to a surface of the hair in need of treatment a protein composition as provided in statements 1-19, the administration resulting in protein treated hair;
administering to a surface of the protein treated hair an activator composition comprising a co-initiator compound and a crosslinker compound as provided in statements 1-19 resulting in activator composition treated hair; and
exposing the activator composition treated hair to a light source, and, optionally exposing the hair treated with the activator composition to heat thereby causing the hair to exhibit less frizz.

Statement 30. The method of statement 29, wherein the hair frizziness is reduced by at least 10%, 20%, 30%, 40%, 50% or more.

Statement 31. The method of statements 29-30, wherein the hair is exposed to the light source for at least one minute.

Statement 32. The method of statements 29-31, wherein the light source emits a light having a wavelength of from about 1 nm to about 750 nm.

Statement 33. The method of statements 29-32, wherein the alpha keratin, gamma keratin, or combination thereof are present in the protein composition in an amount of about 0.01% w/w to about 10% w/w based on the total weight of the protein composition.

Statement 34. The method of statements 29-33, wherein the crosslinker compound is riboflavin-5-phosphate.

Statement 35. The method of statements 29-34, wherein the crosslinker compound and co-initiator compound are each present in the activator composition in an amount of from about 0.01% w/w to about 10% w/w based on the total weight of the activator composition.

Statement 36. The method of statements 29-35, wherein the co-initiator compound is sodium persulfate, L-arginine, or a combination thereof.

Statement 37. The method of statements 29-36, further comprising the step of adjusting light wavelength, light intensity, temperature, duration of light exposure, amount and length of time the activator composition is left in the hair, weight percent of components included in the keratin protein composition and activator composition, or any combination thereof to achieve a desired reduction of frizz.

Statement 38. A method relaxing hair or reducing curl pattern is provided that includes:
administering to a surface of the hair in need of treatment a protein composition as provided in statements 1-19, the administration resulting in protein treated hair;
administering to a surface of the protein treated hair an activator composition comprising a co-initiator compound and a crosslinker compound as provided in statements 1-19 resulting in activator composition treated hair; and
exposing the activator composition treated hair to a light source, and, optionally exposing the hair treated with the activator composition to heat thereby causing the hair to relax and exhibit reduced curl pattern.

Statement 39. The method of statement 38, wherein the light source exhibits a wavelength of from about 400 nm to about 600 nm.

Statement 40. The method of statements 38-39, wherein the light source exhibits a wavelength of from about 445 nm to about 455 nm.

Statement 41. The method of statements 38-40, wherein the light source exhibits an intensity of from about 25,000 lux to about 45,000 lux.

Statement 42. The method of statements 38-41, wherein the activator composition treated hair is exposed to a temperature of from about 25° C. to about 85° C.

Statement 43. The method of statements 38-42, wherein the light source is exposed to the activator composition treated hair for between about 1 minute to about 10 minutes.

Statement 44. The method of statements 38-43, wherein the light source is exposed to the activator composition treated hair for about 1 minute, 2 minutes, 3 minutes, 4 minutes, 5 minutes, 6 minutes, 7 minutes, 8 minutes, 9 minute, 10 minutes, or more to achieve the desired hair curl pattern.

Statement 45. The method of statements 38-44, wherein the co-initiator compound is L-arginine present at from about 1.0% w/w to about 20% w/w based on the total weight of the activator composition.

Statement 45. The method of statements 38-44, further comprising the step of adjusting light wavelength, light intensity, temperature, duration of light exposure, amount and length of time the activator composition is left in the hair, weight percent of components included in the keratin protein composition and activator composition, or any combination thereof to achieve the desired hair curl pattern.

Statement 46. A hair styling photochemical composition is provided that includes:
a keratin protein composition comprising alpha keratin, gamma keratin, or a combination thereof as provided in statements 1-19; and
an activator composition comprising a co-initiator compound and a crosslinker compound as provided in statements 1-19,
wherein upon exposure to light, the photochemical composition is activated.

What is claimed is:

1. A hair styling kit comprising:
a keratin protein composition comprising alpha keratin, gamma keratin, or a combination thereof; and
an activator composition comprising a co-initiator compound and a crosslinker compound,
wherein upon exposure to light, the photochemical composition is activated.

2. The hair styling kit of claim 1, wherein the keratin protein composition further comprises ethylenediaminetetraacetic acid (EDTA) present in an amount of from about 0.01% w/w to about 3% w/w based on the total weight of the protein composition.

3. The hair styling kit of claim 1, wherein the co-initiator compound is sodium persulfate, L-arginine, or a combination thereof.

4. The hair styling kit of claim 1, wherein the crosslinker compound is riboflavin-5-phosphate.

5. The hair styling kit of claim 1, wherein the alpha keratin, gamma keratin, or combination thereof are present in the protein composition in an amount of about 0.01% w/w to about 10% w/w based on the total weight of the keratin protein composition.

6. The hair styling kit of claim 1, wherein the crosslinker compound is present in the activator composition in an amount of from about 0.01% w/w to about 10% w/w based on the total weight of the activator composition.

7. The hair styling kit of claim 1, wherein the co-initiator compound is present in the activator composition in an amount of from about 0.01% w/w to about 30% w/w based on the total weight of the activator composition.

8. The hair styling kit of claim 1, wherein the protein composition includes alpha keratin and gamma keratin.

9. The hair styling kit of claim 1, wherein the alpha keratin to gamma keratin ratio in the keratin protein composition is about 4:1.

10. A method of photochemical hair treatment comprising the steps of:
administering a keratin protein composition comprising alpha keratin, gamma keratin, or a combination thereof, the administration resulting in keratin protein treated hair;
administering to a surface of the keratin protein treated hair an activator composition comprising a co-initiator compound and a crosslinker compound to produce activator composition treated hair; and
exposing the activator composition treated hair to a light source.

11. The method of claim 10, further comprising the step of:
adjusting one or more of: (i) light wavelength; (ii) light intensity; (iii) temperature; (iv) duration of light exposure; (v) amount and length of time the activator composition remains in the hair; (vi) weight percent of components included in the keratin protein composition; and (vii) weight percent of components included in the activator composition.

12. The method of claim 10, wherein the hair is exposed to the light source for at least one minute.

13. The method of claim 10, wherein the light source emits a light having a wavelength of from about 450 nm to about 550 nm.

14. The method of claim 10, wherein the light source exhibits an intensity of at least about 25,000 lux.

15. The method of claim 10, wherein the activator composition treated hair is exposed to a temperature of from about 25° C. to about 95° C.

16. A method of relaxing hair or reducing curl pattern comprising the steps of:
administering a keratin protein composition comprising alpha keratin, gamma keratin, or a combination thereof, the administration resulting in keratin protein treated hair;
administering to a surface of the keratin protein treated hair an activator composition comprising a co-initiator compound and a crosslinker compound to produce activator composition treated hair; and
exposing the activator composition treated hair to a light source thereby causing the hair to relax and reduce curl pattern.

17. The method of claim 16, wherein the hair frizziness is reduced by at least 40%.

18. The method of claim 16, further comprising the step of:
adjusting one or more of: (i) light wavelength; (ii) light intensity; (iii) temperature; (iv) duration of light exposure; (v) amount and length of time the activator composition remains in the hair; (vi) weight percent of components included in the keratin protein composition; and (vii) weight percent of components included in the activator composition.

19. The method of claim 16, wherein the hair is exposed to the light source for at least one minute.

20. The method of claim 16, wherein the light source emits a light having a wavelength of from about 450 nm to about 550 nm.
